SEQUENCE LISTING (3 sequences)

SEQ ID NO: 1 - DNA, 30 bp, synthetic construct
  source 1..30: mol_type = other DNA
tcgtatggcacggggttggtgttgggttgg

SEQ ID NO: 2 - DNA, 16 bp, synthetic construct
  source 1..16: mol_type = other DNA
  modified_base 16: mod_base = OTHER; note = 3-(butyldisulfaneyl)propan-1-ol modified nucleotide
ggtgtgccatacgaaa

SEQ ID NO: 3 - DNA, 16 bp, synthetic construct
  source 1..16: mol_type = other DNA
  modified_base 16: mod_base = OTHER; note = 3-(butyldisulfaneyl)propan-1-ol modified nucleotide
tcgtatggcacaccaa

(19) United States
(12) Patent Application Publication
(10) Pub. No.: US 2024/0377391 A1
(43) Pub. Date: Nov. 14, 2024

(54) Point of Care Device for Detection of Analytes in Tear Film and Other Biological Samples

(71) Applicant: InnSight Technology, Inc., Pittsburgh, PA (US)

(72) Inventors: Leanne Labriola, Pittsburgh, PA (US); Dipanjan Pan, State College, PA (US); Richard W. Mental, Villa Park, IL (US)

(73) Assignee: InnSight Technology, Inc., Pittsburgh, PA (US)

(21) Appl. No.: 18/642,334

(22) Filed: Apr. 22, 2024

Related U.S. Application Data
(60) Provisional application No. 63/497,340, filed on Apr. 20, 2023.

(51) Int. Cl. G01N 33/543; G01N 27/12; G01N 27/30; G01N 27/327

(52) U.S. Cl. CPC G01N 33/5438; G01N 27/12; G01N 27/308; G01N 27/3272; G01N 27/3276; G01N 2333/96486; G01N 2800/162

(57) ABSTRACT

Biosensors for the detection of at least one analyte and osmolarity in a biological sample are provided. The biosensors can comprise a carbon layer, at least one sensor electrode, ligands for the one or more analytes in contact with the carbon layer and/or the at least one sensor electrode, and a substrate.

### PRIORITY

This application claims the benefit of U.S. Ser. No. 63/497,340, filed on Apr. 20, 2023, which is incorporated by reference herein.

### BACKGROUND

Similar to other low volume biological samples, there are many challenges for testing biomarkers in tears. The current standard of care uses lateral flow assays. However, lateral flow assays are particularly affected by low volume, low concentration, and variable viscosity and this interferes with result accuracy in tear film analysis. The requirement to dilute a sample for lateral flow assays, e.g., a tear sample, can result in variability in testing that is seen in current available modalities (InflammaDry®, OrthoQuidel, CA). Currently, lateral flow assays and tear film samples are used for the diagnosis of dry eye disease. Lateral flow assays have been heavily criticized for inconsistent and unreliable results which has limited the acceptance of this by providers and insurance companies to main-stream use. New reliable, point-of-care methods are needed in the art for the detection of analytes in low volume and variable samples, particularly ones that can provide quantitative results.

### SUMMARY

Provided herein are biosensors for the detection of at least one analyte and osmolarity in a biological sample, the biosensor comprising: a carbon layer (e.g., a carbon printed layer), at least one sensor electrode; ligands for the one or more analytes in contact with the carbon layer and/or sensor electrode; and a substrate. The at least one sensor electrode can be present between the carbon layer and the ligands for the analyte. The at least one analyte can be MMP-9 and/or lacritin and the ligands for the one or more analytes can be antibodies or aptamers that can specifically bind MMP-9 and/or lacritin. Osmolarity can be detected in combination with any combination of analytes selected from MMP-9, lacritin, lacroferrin, lysozyme, calgranulin A/B, annexin A1, cystatin S, cathepsin S, PRP4 kinase, tear lipocalin, secretoglobin family 1D member 1 and 2, enolase 1 alpha, mucin MUS5AC, nerve growth factor, human diamine oxidase, calcitonin gene-related peptides, neuropeptide Y, serotonin, epidermal growth factor, interleukins, chemokines, or albumin on the same biosensor or on a different biosensor. The biosensor can comprise a detection area for the at least one analyte and a detection area for osmolarity. The detection area for the at least one analyte and the detection area for osmolarity can be located on the same sensor electrode. The detection area for the at least one analyte and the detection area for osmolarity can be located on different sensor electrodes.

Another aspect provides a method for detecting one or more analytes in a sample and osmolarity of the sample, comprising contacting a biosensor with the sample and detecting the one or more analytes and the osmolarity with a detector. The detector can detect binding or interaction of the one or more analytes and the ligands due to a change in electrical impedance caused by binding or interaction of the one or more analytes in the sample with the ligands. The sample can be tears, tear film, aqueous layer of the tear film, aqueous humor, sweat, blood, serum, plasma, urine, saliva, or other bodily fluids.

Yet another aspect provides a method for detecting one or more analytes in a sample and osmolarity of the sample, comprising contacting the sample with a biosensor, and inserting the biosensor into or onto a device comprising (a) a detector to measure change in electrical impedance, and (b) a screen for visualizing the change in impedance, thereby detecting one or more analytes in the sample and the osmolarity of the sample. The amount of the one or more analytes in the sample can be detected.

Even another aspect provides a method for detecting one or more analytes in a sample and osmolarity of the sample, comprising contacting the sample with a biosensor, wherein the biosensor is present in a handheld device comprising (a) a detector to measure change in electrical impedance, and (b) a screen for visualizing the change in impedance, thereby detecting one or more analytes in the sample and the osmolarity of the sample. The amount of the one or more analytes in the sample can detected.

Another aspect provides a method for diagnosing keratoconjunctivitis sicca (KCS) (dry eye), comprising contacting a biosensor with a tear, tear film, aqueous layer of the tear film, or aqueous humor sample of a subject, detecting an amount of one or more analytes in the sample and osmolarity of the sample, and diagnosing dry eye in the subject where the concentration of the one or more analytes in the sample is elevated or decreased as compared to a control sample or control standard and/or wherein the osmolarity of the sample is elevated as compared to a control sample or control element.

The one or more analytes can be MMP-9 and/or lacritin and an increased amount of MMP-9 and/or decreased amount of lacritin as compared to a control can indicate a diagnosis of dry eye. An increased amount of osmolarity as compared to a control can indicate a diagnosis of dry eye. The one or more analytes can be lacritin, lacroferrin, lysozyme, cystatin S, PRP4 kinase, mucin MUS5AC, calcitonin gene-related peptides, neuropeptide Y, MMP-9, calgranulin A/B, annexin A1, cathepsin S, lacritin, enolase 1 alpha, nerve growth factor, serotonin, interleukins, TNF alpha, albumin or change in epidermal growth factor, secretoglobin family 1D member 1 and 2, tear lipocalin or any combination thereof. The method can further comprise treating the subject with over-the-counter eye drops, over-the-counter moisturizing gels, over-the-counter ointments, prescription drops, cyclosporine (Restasis), lifitegrast (Xiidra), cyclosporine ophthalmic solution, avoidance of smoke, wind, and air conditioning, use of a humidifier, limiting screen time, wearing sunglasses, using tear duct (punctal) plugs, surgery to correct loose lower eyelids, or a combination thereof.

Another aspect provides a handheld device comprising a biosensor for detection of one or more analytes and osmolarity comprising a carbon layer, at least one sensor electrode; ligands specific for the one or more analytes in contact with the carbon layer and/or the one or more sensor electrodes; a substrate; and a detector connected to a data acquisition system. The detector can be a multimeter. The data acquisition system can be a computer, a cell phone, or a tablet. The handheld device can further comprise a screen that allows for visualization of an amount of the one or more analytes and an osmolarity value for the sample. The one or more analytes can be MMP-9 and/or lacritin. The one or more analytes can be lacritin, lacroferrin, lysozyme, cystatin S, PRP4 kinase, mucin MUS5AC, calcitonin gene-related peptides, neuropeptide Y, MMP-9, calgranulin A/B, annexin A1, cathepsin S, lacritin, enolase 1 alpha, nerve growth factor, serotonin, interleukins, TNF alpha, albumin or change in epidermal growth factor, secretoglobin family 1D member 1 and 2, tear lipocalin or any combination thereof. The biosensor can comprise a detection area for the at least one analyte and a detection area for osmolarity. A detection area for the at least one analyte and a detection area for osmolarity can be located on the same sensor electrode. The detection area for the at least one analyte and a detection area for osmolarity can be located on different sensor electrodes.

### BRIEF DESCRIPTION OF THE FIGURES

FIG. 1 shows a testing range for MMP-9. Biosensor chips were able to detect between about 5 ng/mL to about 75 ng/mL of MMP-9 in samples. These biosensor chips can be used in point of care devices as described herein.

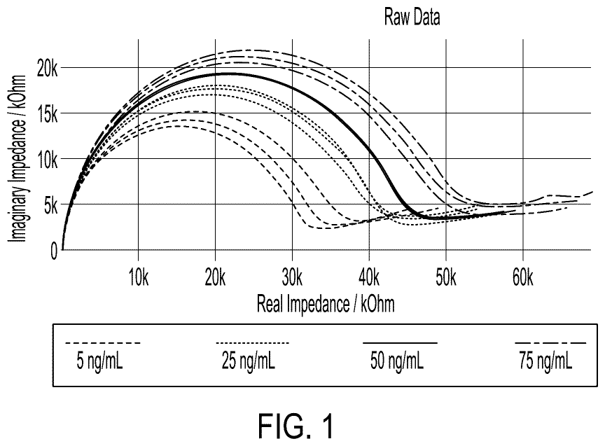

FIG. 2 shows an example of a biosensor chip.

FIG. 3 shows an example of a biosensor chip.

FIG. 4 shows a point of care detector device that can receive a biosensor chip or cartridge.

FIG. 5A to 5C shows examples of detection devices and cartridges containing biosensor chips.

FIG. 6 shows a work-flow including the detector devices described herein.

FIG. 7 shows an example of a work-flow. (1) Tear fluid is collected from the tear lake in the patient at 700, 701 shows the sensor, (2) fluid is placed on the sensor, (3) 702 shows the sensor being inserted into the reader. Optimization of the work steps can allow for direct collection of the tear film of the patient and then the second step is insertion into the base device. 703 shows an example of a circuit of a biosensor chip. The first arrow shows the circuit that measures the electrical change for osmolarity, the second arrow shows the second circuit, which measures the change for, e.g., MMP-9. Additional sensing areas can be included.

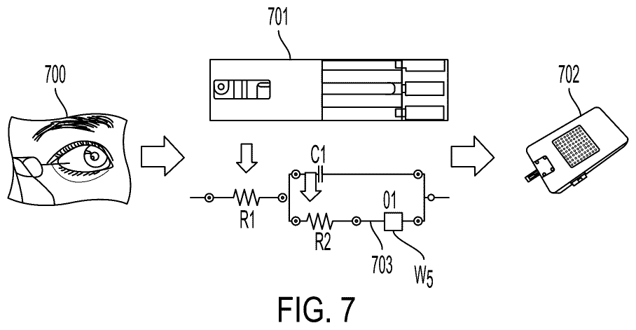

FIG. 8 shows an example of a capillary collection channel on the top of the device, which is an opening to collect fluid in the device sheath. The collection channel is inserted into the fluid (red fluid used for visualization purposes). The fluid flows over the sensing platform. The collection channel is a specialized feature of the device that is designed to collect small volumes of fluid and deliver it to the sensing area.

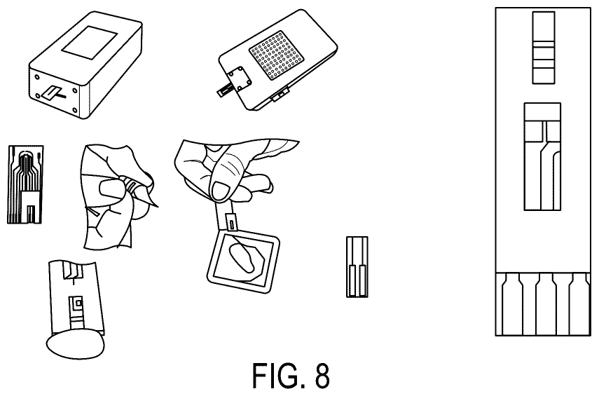

FIG. 9 shows an example of the immobilization chemistry of an antibody to a biosensor chip. The antibody can be used to detect an analyte such as MMP-9.

FIG. 10 shows a comparison of the R2 quantitative resistance measurement of MMP-9 interaction of a device described herein compared to the lateral flow InflammaDry® test in the same patients stratified by exam findings. The top graph compares InflammaDry® positive results with dark result line on lateral flow (+1), semi-positive results with light result line (+0.5), negative result with no line (−1). These results are shown across the clinical exam findings of patients tested. The variable results at all exam levels is consistent with clinician use of this device. The flat fitted line through the graph shows poor disease correlation with InflammaDry®. The bottom graph compares the MMP-9 readings (reported as EIS values) in the same patients to the exam finding. The fitted line shows an upward trend in the results with more severe disease as would be expected. EIS can be converted to concentration. PEE is punctate epithelial erosions which are present with epithelial corneal disease. EIS is electrochemical impedance signal. +1 is a positive reading on a lateral flow device. −1 is a negative reading on a lateral flow device. The R2 value shows a trend toward higher values of MMP-9 with worsening exam findings. The InflammaDry® lateral flow assay provides inconsistent results at each exam level.

FIG. 11 shows MMP-9 concentration in subjects diagnosed as dry eye compared to subjects without the diagnosis of dry eye.

FIG. 12 shows interfering substances. Evaluation of control patient with multiple tear film collections showing the effect of drops on the results obtain. Result of MMP-9 collected after the installation of ophthalmic irrigation solution (BSS) and fluorescein shows significant decline in MMP-9 concentration.

FIG. 13 shows the result of anesthetic (proparacaine) and dilating (tropicamide 2.5% and phenylephrine 2%) medications and artificial tear use on the results of MMP-9 in patients with and without dry eye. The use of the drops affects the MMP-9 concentration particularly in patients with the diagnosis of dry eye.

FIG. 14 shows evaluation of time-of-day effect on concentration of MMP-9.

### DETAILED DESCRIPTION

### Markers and Analytes

Methods and devices provided herein can be used for the detection of one or more analytes within a biological sample, such as tear film or saliva samples. Devices and methods as described herein can be used to detect and quantify one or more analytes such as enzymes, antibodies, proteins, peptides, and nucleic acids in a sample. These analytes can be increased or decreased in amount in subjects having dry eye disease as compared to healthy subjects. Examples of analytes that can be increased or decreased in biological samples as compared to healthy controls or standards are MMP-9 (increased), lacritin (decreased), osmolarity (increased), ascorbic acid (decreased), cortisol (increased), Beta-2-macrogloblulin (decreased), prostate specific antigen (increased), lacroferrin (decreased), lysozyme (decreased), calgranulin A/B (increased), annexin A2(increased), cystatin S (decreased), cathepsin S (increased), PRP4 kinase (decreased), tear lipocalin (decreased), secretoglobin family 1D member 1 and 2, enolase 1 alpha (increased), mucin MUS5AC (decreased), nerve growth factor (increased), calcitonin gene-related peptides (decreased), neuropeptide Y (decreased), serotonin (increased), epidermal growth factor, human diamine oxidase (decreased) and others. Other analytes that can be used include, for example, dehydroascorbic acid, glucose, lactate, uric acid, sialic acid, lactic acid, interleukins, TNF-α, small molecules, immunoglobulins (e.g., IgM, IgG, sIgA), lysozyme, bicarbonates, glucose, lipocalin, and electrolytes (Na⁺, K⁺, Cl⁻, HCO⁻, Mg²⁺, Ca²⁺), lipids (meibomian glands), defensins, collectins, cortisol and dehydroepiandrosterone (DHEA), serotonin, cytokines, inflammatory mediators, growth factors, white blood cells, antigens, signaling molecules, complement components, remodeling enzymes, lypocalin, mucins (epithelial membrane-anchored type, soluble goblet-cell type), albumin, b-FGF, or nucleic acid molecules. In an aspect both an analyte, such as MMP-9, and osmolarity can be detected and quantified using one biosensor.

In one aspect an analyte is present in a biological sample such as tears, tear film, aqueous layer of the tear film, aqueous humor, sweat, blood, serum, plasma, urine, saliva, or other bodily fluids.

A ligand is a substance that forms a complex with an analyte through, for example, ionic bonds, hydrogen bonds, Van der Waals forces, covalent bonds, non-covalent bonds, electrostatic interactions, π-effects, or hydrophobic effects.

A ligand can be immobilized to the sensor (e.g., to the carbon layer and/or the sensor or working electrode) using any suitable technology, e.g., covalent coupling or capture coupling. Immobilization of one or more binding ligands onto a biosensor chip is performed so that the ligand will not be washed away by rinsing procedures, and so that its binding to analytes in a test sample is unimpeded by the biosensor chip surface. Several different types of surface chemistry strategies have been implemented for covalent attachment of ligands to biosensor chips. Surface preparation of a biosensor chip so that it contains the correct functional groups for binding one or more ligands can be an integral part of the biosensor chip manufacturing process.

One or more ligands can be attached to a biosensor chip surface by physical adsorption (i.e., without the use of chemical linkers) or by chemical binding (i.e., with the use of chemical linkers). Chemical binding can generate stronger attachment of specific binding substances on a biosensor chip surface and provide defined orientation and conformation of the surface-bound molecules.

Several examples of chemical binding include, for example, amine activation, aldehyde activation, and nickel activation. These surfaces can be used to attach several different types of chemical linkers to a biosensor chip surface. While an amine surface can be used to attach several types of linker molecules, an aldehyde surface can be used to bind proteins directly, without an additional linker. A nickel surface can be used to bind molecules that have an incorporated histidine (“his”) tag.

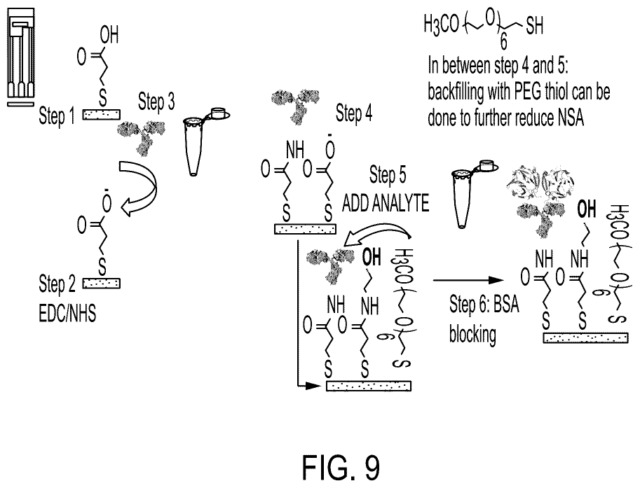

An example of immobilization of an antibody ligand is shown in FIG. 9. In step 1, mercaptohexanoic acid can be dissolved in 50% or lower alcohol and added to the biosensor chip for 3 hours. In step 2 EDC (0.1M) and NHS (0.025M) is added for 30 minutes to activate —COOH groups. In step 3 an antibody ligand, e.g., an antibody that can specifically bind to MMP9 is added and incubated for 30 minutes. Ethanolamine can be added for 15 minutes in step 4. Between steps 4 and 5 PEG thiol can be added to reduce NSA. MMP-9 or a sample is added to the biosensor chip and incubated in step 5 for 15-30 minutes. The last step can include BSA blocking.

The interaction of ligands with their analytes can be characterized in terms of a binding affinity. For example, a ligand can bind an analyte with a K_{d} equal to or less than about 10⁻⁷ M, such as but not limited to, 0.1−9.9×10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵ or any range or value therein. A ligand can bind an analyte with an off rate (Kott) of less than or equal to 0.1−9.9×10⁻³ sec⁻¹, 10⁻⁴ sec⁻¹, 10⁻⁵ sec⁻¹, 10⁻⁶ sec⁻¹, 10⁻⁷ sec⁻¹. A ligand can bind an analyte with an on rate (Kₒₙ) greater than or equal to 0.1−9.9×10⁻³ M⁻¹ sec⁻¹, 10⁴ M⁻¹ sec⁻¹, 10⁵ M⁻¹ sec⁻¹, 10⁶ M−′sec¹, 10⁷ M⁻¹ sec⁻¹, 10⁸ M⁻¹ sec⁻¹.

A ligand for an analyte can be, for example, an antibody (or specific binding portion thereof) that specifically binds the analyte, an antibody fraction, a nucleic acid, a protein, a peptide, a peptidomimmetics, an ion, a small molecule, an enzyme, or an aptamer. Specifically binds means that a ligand reacts or associates more frequently, more rapidly, with greater duration and/or with greater affinity with a particular target analyte than it does with alternative unrelated target analytes.

A ligand for ascorbic acid (AA) can be, for example, ascorbate oxidase, or an antibody that specifically binds ascorbic acid. A ligand for AA can also be a dual molecule comprised of a fluorescent chromophore and a nitroxide radical (the nitroxide acts as a quencher of the fluorescence of the chromophore fragment). The reduction of the nitroxide fragment by ascorbic acid results in decay of ESR (electron spin resonance) signal and enhancement of the fluorescence.

The ligand for the analyte can be an enzyme. In one aspect, the enzyme is ascorbate oxidase, which can be present in a biosensor at a concentration at about 3, 5, 10, 12, 15, 20, 30, 40, 50, 60, 70, 80, 90, 100 or more U/cm².

Examples of ligands include, for example: synthetic lectins for glucose (Ke et al., Nature Chemistry, D01:10.1038 NCHEM.1409); 5-amino-2-hydroxymethylphenyl boronic acid for lactate; polymers for uric acid (US Publ. 20030039627); lectins for sialic acid (Zeng & Gabius, Z. Naturaforsch. C. (1992) 47:641); trivalent chromium and dextran for lactic acid; antibodies, interleukin receptors, or binding portions thereof for interleukins; antibodies, TNF-α receptors, or binding portions thereof for TNF-α; antigens for immunoglobulins (e.g., IgM, IgG, sIgA); anti-σ factor RsiV and dapsone for lysozyme; carbonic anhydrase and phosphoenolpyruvate carboxylase for bicarbonate; Pneumococcal surface protein A, GAPDH, osteopontin, and DNA for lactoferrin; enterobactin for lipocalin; heparin, Na⁺/K⁺ ATPase, glycine, polystrene sulfonate resin, valinomycin, aequorin, chromomycin3, norfloracin, cholera toxin, troponinC, S100A1 binding protein and calsequestrin for electrolytes (Na⁺, K⁺, Cl⁻, HCO⁻, Mg²⁺, Ca²⁺); Staphylokinase and heparin for defensins; Gp340 and calreticulin for collectins; corticosteroid binding globulin for cortisol; MAP2 for dehydroepiandrosterone (DHEA); seronectin for serotonin; cytokine receptor proteins or binding portions thereof for cytokines, GroEL, GAPDH from Mycoplasma genitalium, LL-37 peptide for mucins; FcRn, Protein G, and albondin for albumin; syndecan, PG-M-CSF, and perlecan for b-FGF. Additionally, ligands in the form of antibodies and specific binding portions thereof are well known to those of skill in the art for the above-mentioned analytes.

Markers and analytes can be detected using aptamers that specifically bind the markers or analytes. Aptamers are three-dimensional oligonucleotides (RNA or single-stranded DNA structures) that can bind with high selectivity and specificity to markers and analytes due to their spatial conformation. In some aspects, aptamers can provide for advantages over the use of antibodies, including greater thermal stability which extends their shelf-life and aptamer conformation-recovery properties that make aptamer-based sensors reusable.

In an aspect, an analyte can be an antibody that specifically binds a biomarker, e.g., MMP-9. Levels of MMP-9 can change from 5 ng/ml to 200 ng/ml in different disease states, but certain current assays only provide a cut off value of positive when above 40 ng/ml. This is insufficient to adequately diagnosis patients or follow disease or treatment response. Antibodies that bind MMP-9 include, for example, AF911 and MAB936 from Biotechne, AB19016 from Sigma Aldrich, and MA515886 from Invitrogen among others. Aptamers that specifically bind MMP-9 include, for example, TCG TAT GGC ACG GGG TTG GTG TTG GGT TGG-3′ (SEQ ID NO:1); comp-1 of MMP-9 aptamer, 5′-GGT GTG CCA TAC GAA A/3ThioMC3-D/-3′ (SEQ ID NO:2); comp-2 of MMP-9 aptamer, 5′-TCG TAT GGC ACA CCA A/3ThioMC3-D/-3′ (SEQ ID NO:3). /3ThioMC3-D/stands for 3-(butyldisulfaneyl)propan-1-ol to provide a disulfide structure at the 3′ end of the DNA strands. See, e.g., Kim et al., Photoacoustics, Volume 25: 100307 (2022).

In an aspect, an analyte can be an antibody that specifically binds a biomarker, e.g., lacritin. Antibodies that bind lacritin include, for example, 18271-1-AP from ProteinTech or LS-C314921 from LSBio.

In one aspect a ligand for an analyte is stable at room temperature (about 20 to 22° C.), field temperatures (about 0 to 49° C.), refrigeration temperature (about 1 to 5° C.), or freezing temperature (about 0 to −70° C.) for 1 day, 2 days, 3 days, 4 days, 5 days, 1 week, 2 weeks, 3 weeks, 4 weeks, 2 months, 3 months, 6 months, 1 year, 2 years or longer.

Existing methods of detecting osmolarity of a sample provide a quick result but are limited because of high error rates with the lower sample of volume from tear film collection. In a research setting, the most commonly applied laboratory technique for measurement of tear osmolarity is through observation of the change in the freezing point of tear samples. The technique, however, includes the need for specialist expertise, constant maintenance of equipment, a significant amount of time, a large laboratory setup, and there may be errors due to evaporation of the test sample or control standards. The instant compositions and methods can accurately detect osmolarity of small sample volumes (e.g., tear film) using resistance or impedance readings at a location on a biosensor chip that can be present in a point of care device as described herein.

### Biosensor Chips

A biosensor chip or biosensor can comprise an electrode printed or placed on a carbon layer. Carbon atoms can possess various physical structures with distinct physical attributes. A carbon layer can be made up of carbon spheres and ellipsoids, carbon nanotubes (CNTs), nanohorns (CNHs), graphene, fullerene, carbon dots (CDs), carbon nanodiamonds (CNDs), graphene quantum dots (GQD), single-walled carbon nanotubes (SWNTs), multiwalled carbon nanotubes (MWCNTs), or cup-stacked carbon nanotubes (CSCNTs), graphite, and carbon nanofibers (CNFs), carbon sponges felt. A detection layer (e.g., an enzyme, protein, aptamer, or antibody layer) can be present on the electrode. A barrier layer can cover the detection layer. An electrode is a composition that, when connected to an electronic device, is able to sense a current or charge and convert it to a signal. Alternatively, an electrode can be a composition that can apply a potential to and/or pass electrons to or from connected devices.

Electrodes include, but are not limited to, certain metals and their oxides, including gold; copper; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite and carbon paste). In one aspect, the electrode can be an interdigitated electrode, micro-interdigitated gold electrode (MICE), or a digitated electrode (e.g., a digitated gold electrode).

In one aspect, gold is deposited on a biosensor electrode at a thickness of about 50,100,200, 300, 400, 500, or more nm.

The electrode can be a planar electrode. The electrode can be deposited on a biosensor by a variety of methods including, but not limited to, screen-printing or evaporation. An electrode can be open or covered by a cover to form a defined volume cell.

A biosensor electrode can detect a change in resistance or impedance caused by the interaction of a ligand and an analyte such as an antibody or aptamer specific for MMP-9 or lacritin. Reactance is the resistance (R) offered to AC current by inductors and capacitors. Impedance (Z) is the sum of the resistance (R) and reactance. The change in resistance or impedance can indicate an amount of the analyte present in the sample. A biosensor electrode can also detect a change in resistance or impedance caused by the osmolarity of the sample. The change in resistance or impedance can indicate an amount of osmolarity of a sample.

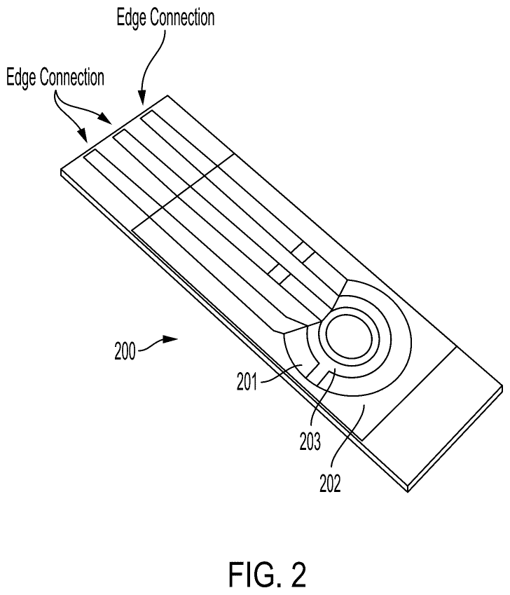

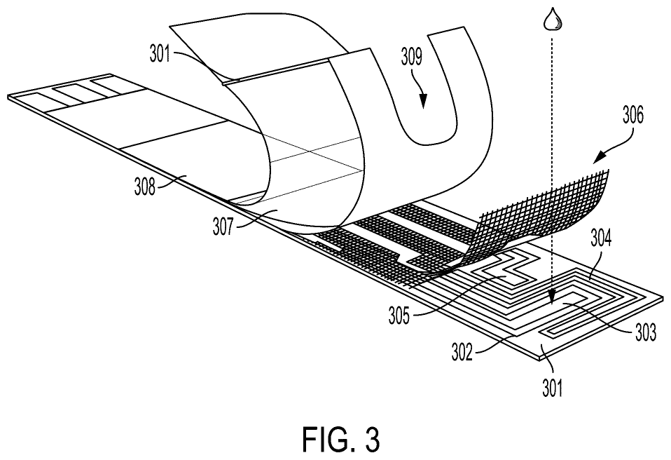

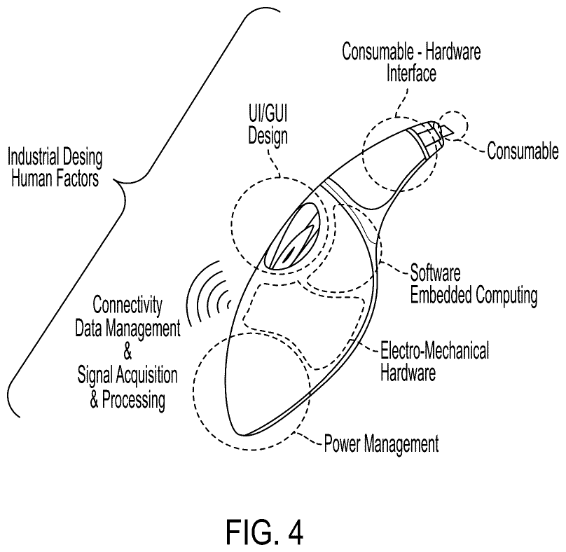

In an aspect, a working or detector electrode 200, a reference electrode, and a counter electrode can be present on a chip. See, e.g., FIG. 2. A reference electrode 201 can allow for the measurement of the potential of the working or sensor electrode 203 without passing current through it while a counter electrode 202 can allow for passing current. A biosensor chip can be configured to fit in a device that can be used to collect a sample and analyze the sample. See FIG. 4. A biosensor chip can have a height of about 0.3, 0.4, 0.5, 0.6, 0.635, 0.7, 0.8, 1.0 mm or more. A biosensor chip can have a length of about 20, 25, 30, 35, 40, 45, 50 mm or more. A biosensor chip can have width of about 5, 6, 7, 8, 9, 10, 11, 12, 13, 14, 15 mm or more. A detector device can comprise a slot to insert a consumable (i.e., one or more biosensor chips or cartridges). In an aspect a chip can comprise a base layer such as polyester, plastic, or polymer base. See FIG. 3. A base layer can comprise one or more of an acrylamide, cellulose, nitrocellulose, glass, indium tin oxide, silicon wafer, mica, polystyrene, or polyvinylidene fluoride (PVDF) filter, filter paper (e.g., VVhatman), glass fiber filters (GF), fiberglass, polyethylimine coated GFs, porous mylar or other transparent porous films, cellulose nitrate (CN) membrane, mixed cellulose ester membrane, cellulose acetate membrane, polyethersulfone (PES) membrane, PTFE membrane, ultrafiltration membranes of poly(vinyl chloride) (PVC), carboxylated poly(vinyl chloride) (CPVC), polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamine acids. The base layer can be formed into pre-perforated strips, individual strips, individual sheets, or any other suitable shape. A carbon layer 302, e.g., a carbon printed layer can be present on the base layer 301. A working or sensor electrode 303 can be present on the carbon layer and can comprise one or more detection ligands (e.g., antibodies, enzymes, proteins, or aptamers). The one or more ligands can be layered and/immobilized on the working or sensor electrode on one or more portions of the electrode or over the entire electrode. A layer of ligand is not necessarily contiguous over the working or sensor electrode depending on the methodology used to apply the ligands to the electrode. That is, the ligands can be spaced from each other on the electrode and still be considered a layer of ligands. A reference electrode 304 can be present on the biosensor chip and can be, for example, silver and/or silver chloride. In an aspect two more resistance or impedance values can be present on the same circuit so that two of more properties of the test fluid can be tested. A fill trigger electrode 305 can also be present on the carbon layer and can be silver and/or silver chloride or other suitable material. The carbon layer can provide one or more conductive tracks or microchannels (e.g., 1, 2, 3, 4, 5 or more) for the working, reference, and trigger electrodes. A sample can be delivered to the conductive tracks or microchannels which lead to the electrodes. More than one working or sensor electrodes (e.g., 1, 2, 3, 4, 5, or more) can be present to detect and quantify more than one analyte at a time. A biosensor can comprise a detection area for the at least one analyte and a detection area for osmolarity. A detection area for the at least one analyte and the detection area for osmolarity can be located on the same sensor electrode. A detection area for the at least one analyte and the detection area for osmolarity can be located on different sensor electrodes.

A mesh layer 306 can be present on the electrodes to support transport of a biological sample to the sample measurement chamber at the working or sensor electrode. An insulation print layer 307 can hold the mesh layer in place, insulate the circuitry, and define the sample chamber. A tape layer 308 on top of the insulation print layer can provide protection from environmental and physical effects. This tape layer can be semi-opaque or clear to allow for the visual confirmation of the samele fill. An air bleed thread 310 can be present in the tape layer to allow the sample chamber to fill.

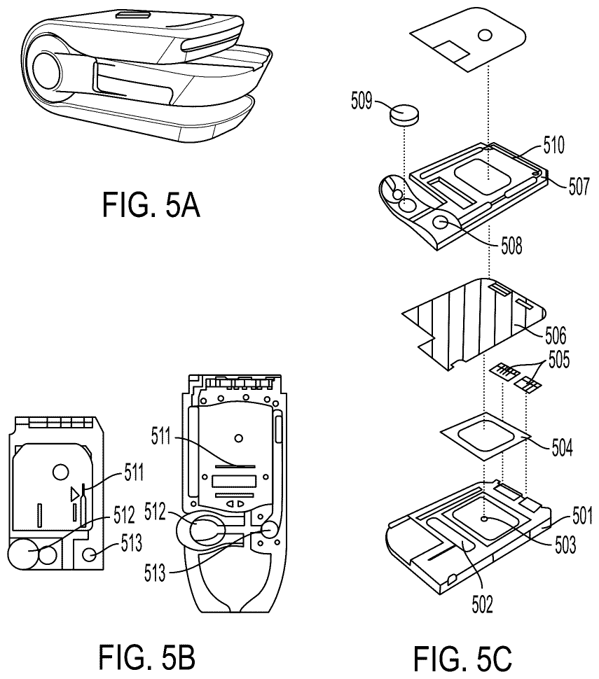

Another aspect provides a cartridge including one or more biosensor chips. See FIG. 5C. A cartridge can comprise a base 501 including an air bladder 502 and a puncturing barb 503 for an optional calibrant pouch (which can include any calibrants or reagents) 504. A cartridge label can be present 590. One or more biosensor chips (e.g., 1, 2, 3, 4, 5, or more) can be present on the cartridge base and are covered by a tape gasket 506. A cartridge cover 507 can be present on the tape gasket. A cartridge cover can comprise one or more microfluidic channels 510 (e.g., 1, 2, 3, 4, 5 or more), a sample entry well 508, and a sample entry well gasket 509. The cartridge can comprise a sample fill mark 511, a tab for snap closure 512, and a sample well 513. See FIG. 5B.

One or more cartridges or biosensor chips can be disposable and can fit within a sensing device or detector.

### Detectors

A detector device can be an electrochemical sensor, e.g., an amperometric, potentiometric, impedimetric, photoelectrochemical, and electrogenerated chemiluminescence sensor. Amperometric sensors have a voltage placed between reference and sensor or working electrodes to cause electrochemical oxidation or reduction. The current is measured as a quantitative indicator of the analyte's concentration. In potentiometric sensors when specific sensor-analyte interactions occur, a local Nernstian equilibrium is formed at the sensor interface which can be used to calculate the analyte's concentration. Impedimetric sensors or conductometric sensors measure changes in the surface impedance to detect and quantify analyte-specific recognition events on the electrode.

In an aspect, impedance based biosensor detection comprises application of a small amplitude AC voltage to the sensor electrode and measurement of the in/out-of-phase current response as a function of frequency. Impedance biosensors can be fabricated by immobilizing a ligand onto a conductive and biocompatible electrode and then detecting the change in the interfacial impedance upon analyte binding.

One or more biosensor chips or cartridges can be used with a detector device. A detector device can be handheld (i.e., less than 12 inches in length and 2, 1, 0.75, 0.5 pounds or less in weight). A detector device can be connected to a data acquisition system. A detector device can include an integrated sample collection element. A detector can comprise a digital or analog multimeter that can measure voltage, current, impedance and/or resistance. A detector can also be a spectrophotometer, fluorometer, or a spectrometer like a Raman spectrometer or a Fourier transform infrared spectrometer.

A data acquisition system can be, e.g., a computer, a hand-held device, a cell phone, and/or a tablet. The detector provides information (e.g., a sample identifier, a subject identifier, a quantity detected of one or more analytes, an osmolarity reading, a positive or negative reading regarding the presence or absence of an analyte, or a combination thereof) to the data acquisition system, which can then analyze the information and provide an easy to read and interpret result.

A device can further comprise a screen that allows for visualization of an amount of an analyte, e.g., MMP-9, lacritin, or ascorbic acid, present in a sample and/or the osmolarity of a sample. A device can be battery operated. Results can be time stamped to avoid any issues with diurnal curve. The devices can be Bluetooth capable to transfer data directly into an electronic medical record.

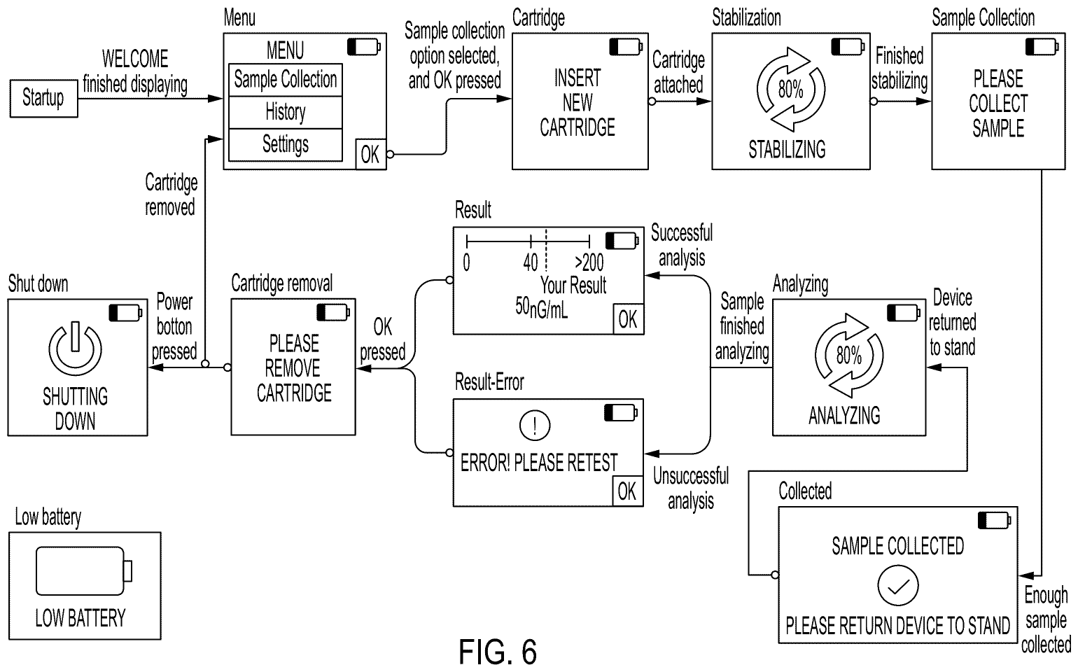

FIG. 6 shows an example of a work-flow using a device as described herein. A device can be turned on. A menu can be displayed including selections such as sample collection, history, and settings. Where sample collection is selected the device display instructs a user to insert a new cartridge. The device can then go through a stabilization mode in preparation for sample collection. In an aspect the stabilization is where the impedance reaches a stable state. The device display can then instruct the user to collect a sample, e.g., a tear film. Once the device receives enough sample, it can instruct the user that the sample has been collected. The device can ask the user to return the device to a stand to start analyzing the sample. The device can then analyze the sample. The device display can then show the result and show where in a range the sample falls (e.g., normal or elevated or depressed). The device can display a result in, e.g., pg or ng/mL and show how the result falls into a range. If the analysis is unsuccessful an error result can be displayed that instructs the user to retest. The device display can then indicate to the user that the cartridge can be removed. The device can then be turned off. In an aspect, a low battery screen can be displayed when the battery power is low.

Also provided are kits comprising one or more biosensors or cartridges, one or more disposable biosensors or cartridges, a detector, a data acquisition system, or combinations thereof.

### Methods of Detection of Analytes and Diagnosis

In an aspect the devices and methods provide 60% less processing time and greater sensitivity (70% compared to 30%) than other tests, e.g., InflammaDry®. In an aspect, results can be delivered in about 10, 7, 5, 4, 3, 2, or less minutes. The instant methods and devices allow for direct tear collection (without dilution) and microfluidic control. Microfluidic channels can be reduced in size to accept and process as little as 1 μL of fluid (e.g., about 50, 40, 30, 20, 10, 5, 4, 3, 2, 1, 0.5, 0.1, 0.05 μL or less).

Methods are provided for detecting a dry eye condition in a subject comprising detecting the level or amount of an analyte (e.g., MMP-9 and/or lacritin) and an osmolarity level in a biological sample (e.g., tear film) from a subject. An elevated level of MMP-9 and/or a decreased level of lacritin and/or an elevated osmolarity can indicate an eye condition, e.g., a dry eye condition, is present in the subject. The level or amount of MMP-9 and/or lacritin and/or osmolarity in a tear film can be compared to a control sample or control standard. If an elevated level of MMP-9 and/or a decreased level of lacritin and/or an elevated osmolarity is present in the tear film, then a medical practitioner can administer one or more treatments (e.g., administration of medication, etc.) to the subject.

In an aspect, the detection of osmolarity and/or one or more analytes as described herein can be used to detect and/or diagnose meibomian gland disease, pterygiums, conjunctival chalasis, allergic conjunctiva, corneal ulcers, and glaucoma.

Also provided are methods of detecting an analyte such as MMP-9 and/or lacritin in combination with osmolarity in a sample comprising contacting a biosensor chip or cartridge with the sample and detecting the analyte with a detector.

The detector can detect binding of the analyte and the analyte ligand due to a change in electrical resistance or impedance caused by interaction of the analyte in the sample with the analyte ligand; due to a change in mass on the biosensor; due to a colorimetric change; due to a fluorescent reaction; due to a change in a Raman spectroscopy reading, or due to a change in a Fourier transform infrared spectroscopy reading. Osmolarity can also be detected in this manner. In one aspect, after contacting a biosensor chip or cartridge with a sample, the biosensor is inserted into or onto a device comprising a detector to measure a change caused by interaction of an analyte in the sample and a ligand for the analyte or osmolarity such as a change in electrical resistance or impedance. A screen for visualizing the change in resistance or impedance is provided such that an analyte in the sample is detected.

The presence, absence, or an amount of an analyte in the sample along with osmolarity can be detected. In an aspect, a device can detect about 100, 90, 75, 60, 50, 40, 30, 20, 10, 5 ng/mL or less of an analyte, such as a protein, in a sample. The amount of an analyte, such as MMP-9, can indicate the severity of the eye condition, e.g., a dry eye condition. That is, the higher the amount of analyte, e.g., MMP-9, or osmolarity the greater the severity of the disease or condition (e.g., a dry eye condition). In an aspect, a device can detect about 100, 90, 75, 60, 50, 40, 30, 20, 10, 5 ng/mL or less of MMP-9 in a sample. In an aspect more than about 35, 40, or 45 ng/mL of MMP-9 can indicate dry eye. In an aspect abnormal tear osmolarity can be about 308, 310, 312, 314, 316, 318, 320 or more mOsm/L in one eye, or a difference of greater than about 7, 8, 9 10 or more mOsm/L between the eyes.

Methods of diagnosing an eye condition, e.g., dry eye, are provided. The methods comprise contacting a biosensor chip as described herein with a biological sample, e.g., a tear; tear film; aqueous layer of the tear film, or aqueous humor sample of a subject and detecting an amount of an analyte and/or osmolarity in the sample. An eye condition is diagnosed in the subject where the concentration of the analyte in the sample is elevated as compared to a control sample or control standard and/or the osmolarity is elevated as compared to a control sample or control standard.

Methods and compositions as described herein can provide specificity i.e., the ability of an assay to measure a specific analyte, e.g., MMP-9, rather than other analytes in a sample. The specificity can be, for example, 70, 75, 80, 90, 95, 96, 97, 98; 99% or more. Methods and compositions described herein can provide 10, 9, 8; 7, 6, 5, 4, 3, 2, 1% or less false positive results.

In one aspect, a biosensor chip can be used to detect analytes such as ascorbic acid or MMP-9 and/or osmolarity in other types of samples such as sweat, blood, serum, plasma, urine, saliva, or other bodily fluids to diagnose or detect other conditions.

Analytes such as MMP-9 can be found in higher amounts or levels in injured or diseased samples (e.g., a sample of a subject with an eye condition) as compared to control subject samples from non-injured or non-diseased subjects. The relative levels of analytes, such as MMP-9, in subject samples can indicate progression of disease and disease severity. That is, in some instances, a greater amount or level of analyte in a test sample means a more severe disease state or condition.

In some aspects, the level of analytes such as MMP-9 or osmolarity in a test sample is compared the level of the analyte or osmolarity in a control sample from one or more normal control subjects. Typically, the measured control level in the control sample is then compared with the analyte level or osmolarity measured in the test sample. Alternatively, the level of an analyte such as MMP-9 or osmolarity level in the test sample is compared to a previously determined or predefined control level (a “control standard”). For example, the control standard for an analyte such as MMP-9 can be calculated from data, such as data including the levels of the analyte in control samples from a plurality of normal control subjects. The normal control subjects and the test subject under assessment can be of the same species.

### Treatment

Treatments for dry eye include, for example, over-the-counter eye drops, over-the-counter moisturizing gels, over-the-counter ointments, cyclosporine (Restasis), CEQUA© (cyclosporine ophthalmic solution), lifitegrast (Xiidra), Lacripep® (Tear Solutions), avoidance of smoke, wind, and air conditioning, use of a humidifier, limiting screen time, wearing sunglasses, using tear duct (punctal) plugs, and surgery to correct loose lower eyelids.

In an aspect, a method for detecting an analyte in a sample is provided. The method comprises contacting a biosensor as described herein for the detection of an analyte, e.g., MMP-9, lacritin and osmolarity in a biological sample. The analyte and osmolarity are detected with a detector. Where the patient is diagnosed with dry eye, the patient can be treated with one or more dry eye treatments.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### Examples

### Example 1 Methods

A prospective, masked, controlled study was conducted that evaluated the results of MMP-9 and osmolarity testing with an impedance-based sensor (see FIG. 2) Thirty-two samples from 18 individuals diagnosed with dry eye and 15 samples from 9 control individuals were collected during a routine office visit at a private multi-specialty ophthalmology practice in western Pennsylvania. Inclusion criteria included: age greater than 18 years. Subjects were excluded if they had an active ocular infection of the anterior surface of the eye, corneal abrasion, or eyelid deformity that would prevent tear collection. An impedance-based biosensor as described herein was compared to the standard of care devices (tear osmometer for osmolarity (TearLab Corp, USA); lateral flow testing for MMP-9 (InflammaDry®, QuidelOrtho, USA), using tear film samples collected from clinical patients. Samples were collected using a standard capillary tube after informed consent was obtained. The tenants of the Health Insurance Portability and Accountability Act were followed.

The average age for the dry eye subjects was 73 years old (40-91) compared to 61 (25-80) in the non-dry eye group. Fifteen female and 3 males were in the dry eye group compared to 3 females and 6 males in the control group. Eighteen percent of the control group was being seen in clinic for post-operative evaluation after cataract surgery.

The impedance-based sensor provided a simultaneous reading of quantitative MMP-9 and osmolarity on the same platform. Investigational Device Exemption status was obtained to use the sensor for analysis.

### Example 2 Protocols

Tear film samples were collected without administration of topical anesthetic in order to not alter the tear film. An average of 2 microliters (μl) of fluid was collected by a trained member of the research team using a sterile capillary tube. The capillary tube was then frozen at −40 degree Celsius and shipped to the testing facility. The samples were defrosted and tested on the sensor.

Data collected included age, gender, time of collection, time of freezing and time of testing, drops administered prior to collection, current ocular medication and diagnoses, symptoms, and exam findings. Patient tear film was tested using the commercially available later flow test for MMP-9, and with the osmometer for osmolarity. Patient tears were also collected and tested with the impedance sensor for measurements both quantitative MMP-9 and osmolarity.

A separate collect was done on the same subject three different times to estimate the effect of diluting drops prior to collection. These replicate samples were excluded from the MMP-9 comparison compared to exam severity but included in the evaluation of topical drops on final MMP-9 results. All samples were collected on the same day. The time of collection was noted and used for analysis of concentration compared to time of day.

The manufacturer's instructions were followed for the commercially available devices. The technicians who performed the testing for with the commercially available devices were masked to the exam findings of the patient and the study results of the biosensor.

Analysis was carried out by comparing the MMP-9 results across different instruments. The results from the quantitative MMP-9 test are referred to as (R2 values). The R2 results were compared between subjects diagnosed with and without DED. A p-value of <0.05 was considered statistically significant throughout the study. Clinical diagnosis for each patient was confirmed during the collection allowing for the use of sensitivity and specificity analysis for each device. A tear osmolarity value of 305 mOsm/L was used as the cutoff for mild dry eye, and 316 mOsm/L was used as the cut-off for more severe dry eye disease. Statistical analyses were performed with commercial software JMP Pro 15.0 and visualizations provided using Tableau 2021.4.

### Example 3 Results

Twenty-seven subjects met inclusion criteria, and 47 samples were tested. Forty-seven samples were tested with the impedance sensor, 23 samples with the osmometer and 21 with the lateral flow test for MMP-9. Data from 20 samples (43%) were compared across all three devices. The mean age of the patient group was 73 years old, with ages ranging from 40 to 91 years old and included fifteen females and three males. The mean age of the control group was 61 years old and included three females and six males. Four samples from the control group were collected during a post-cataract surgical follow up visit.

The impedance sensor had a sensitivity of 71% which was more than double the other devices for measuring elevated markers in patients with DED in 20 of the 28 samples. The osmometer showed that all measurements were under the cut off value for DED making the sensitivity 0% for 0 of the 17 DED samples tested. The lateral flow device had a sensitivity of 37.5% finding 6 out of the 15 DED subjects with positive results. The specificity for the novel sensor was 57%; 8 of 14 samples were identified with no markers and no diagnosis of DED. Specificity of the other devices was 100% having all 5 of the clinically undiagnosed not identified (Table 1).

TABLE 1
Osmolarity cutoff values and Device Specificity

| | Sensitivity | Specificity | Sample size |
|---|---|---|---|
| Impedance Base Sensor | 0.71 | .57 | 42 |
| (ROC cutoff value >30900) | | | |
| Osmometer | 0.00 | 1.00 | 23 |
| Lateral Flow MMP-9 | 0.37 | 1.00 | 21 |

### Osmolarity Testing

Twenty-two samples (47%) were tested for osmolarity with both the osmometer and the impedance sensor of who 17 had a known DED diagnosis. The range of osmolarity in the control group was 209 to 292 and in the experimental group was 274 to 303 using the osmometer. In patients with known diagnosis of DED, the osmometer reported an elevated reading in 0 samples (0%) and the impedance sensor reported elevated osmolarity in 13 samples (76%). Three samples without the diagnosis of DED had elevated osmolarity using the impedance sensor.

### Matrix Metalloproteinase Testing

Twenty samples were tested with both the lateral flow and impedance-based sensor for MMP-9, fifteen of which had a DED diagnosis. In DED subjects, the impedance sensor measured an elevated resistance for MMP-9 in 6 samples (40%) and the lateral flow assay was positive in 5 samples (30%), which included one (6.7%) weekly positive and three (20%) mildly positive on the qualitative test.

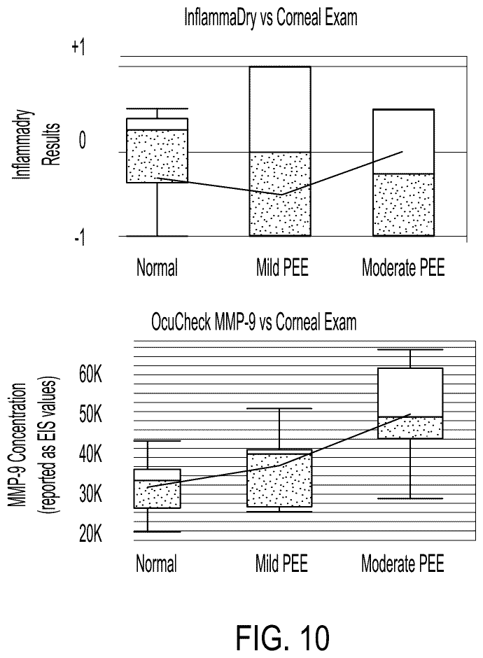

In regard to patients with positive exam findings, the impedance-base sensor showed improved correlation to findings of epithelial breakdown as compared to the lateral flow assay. The quantitative resistance measurements of MMP-9 signal with the impedance sensor (R2) were elevated in 11 samples (73%) with abnormal corneal exam findings on the day of collection. FIG. 10 shows the results of qualitative and quantitative MMP-9 testing compared to corneal exam findings specifically of epithelial breakdown. There was no statistical significance in MMP-9 levels compared to severity of exam findings with either device. However, R2 trended toward higher values as severity of exam findings increased; whereas the lateral flow test had inconsistent results across the severity spectrum.

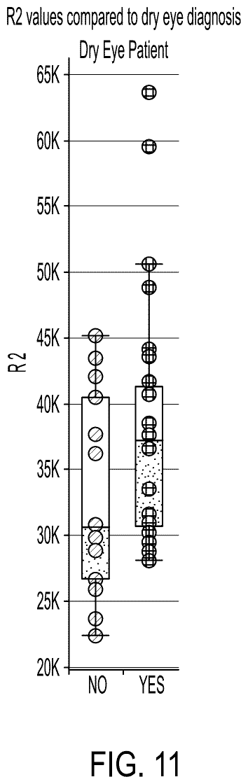

The impedance sensor showed higher R2 values in the dry eye patients compared to those without the disease. (FIG. 11). This coincided with a logistic regression to identify and assess an initial R2 cut off point (30,900) for diagnosis (Chi Squared p-value 0.0871). The MMP-9 measurement using the impedance sensor showed elevated resistance measurements for MMP-9 in 2 out of 5 samples that had no clinical diagnosis.

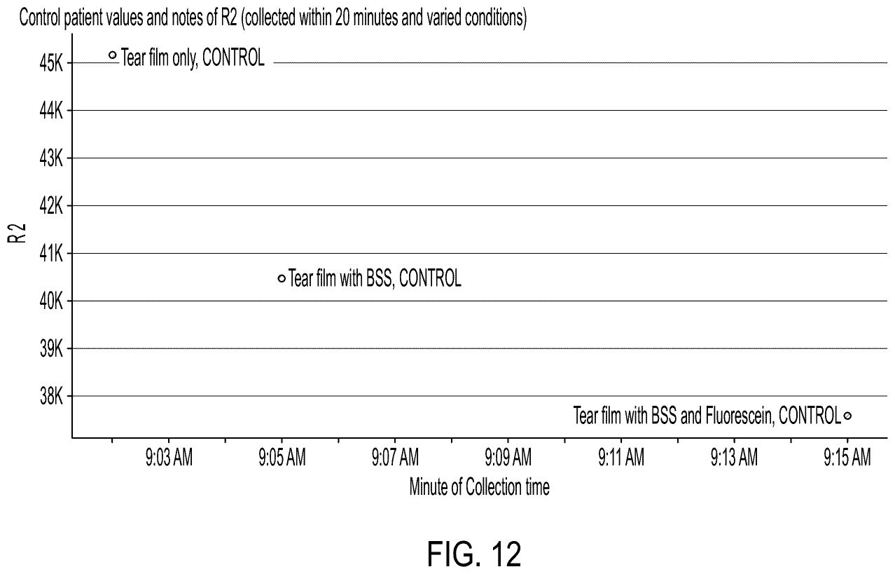

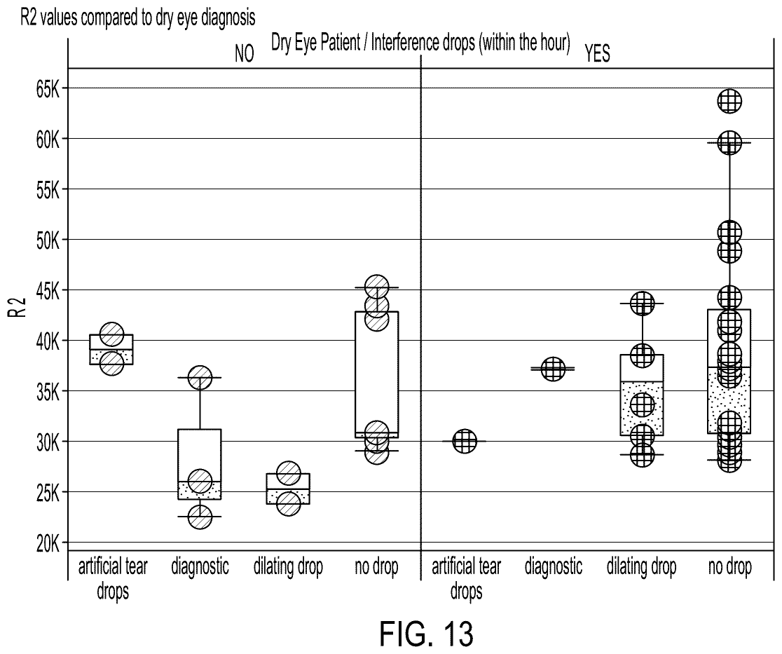

To evaluate the effect on topical application of drops in clinic, R2 in a control patient's tear film showed significant decline after installation of ophthalmic irrigation solution (BSS), and further decline after Fluorescein drops. (FIG. 12). Some patients received anesthetic (proparacaine), dilating (tropicamide 1% and phenylephrine 2.5%) medications, and artificial tears prior to sampling. The effect of these solutions on quantitative MMP-9 values were compared between subjects with and without dry eye disease (FIG. 13).

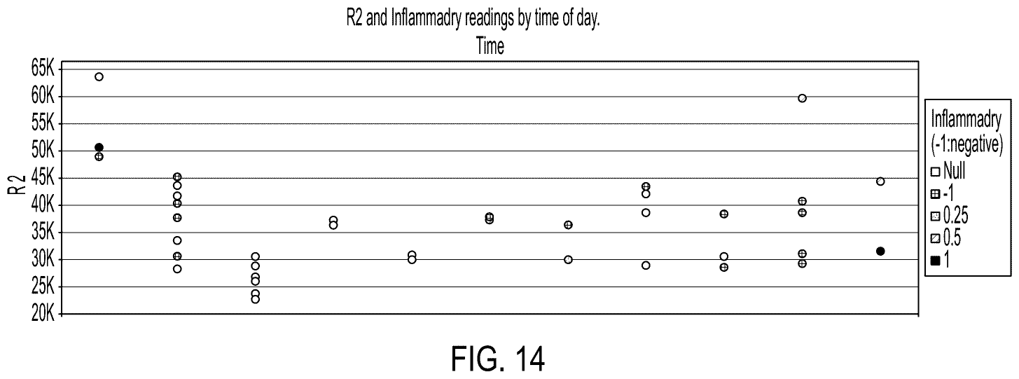

FIG. 14 shows the values of MMP-9 concentration based on time of day. R2 readings revealed higher values in the morning, trended down midday, and then back up again during the later hours of the day.

### Example 4 Discussion Tear Osmolarity and MMP-9 Testing

Tear film testing for biomarkers introduces a new standard for ophthalmic diagnosis. The pathophysiology of MMP-9 activity shows increased expression with epithelial cell turnover and with inflammation of the ocular surface. High clinical correlation of symptoms with elevated MMP-9 validates this enzyme as a biomarker for disease activity and a device was developed for use for commercial testing. New challenges were introduced with understanding the variability of the measurements from the use of lateral flow devices, but limitations in the reproducibility, limit of detection, sensitivity, accuracy, and interpretation bias have constrained the full reach of MMP-9 testing in routine clinical care as well as the ability to expand MMP-9 testing to other indications. Most of the limitations are due to the base technology of lateral flow which is impacted by the tear viscosity, volume, duration of collection and concentration. A cross-sectional study of 188 patients with DED showed that the results obtained from use of the lateral flow device were influenced by the subjects' tear meniscus height (TMH), tear meniscus area (TMA), and tear meniscus depth (TMD) (p=0.033. p=0.017, p=0.010, respectively).²⁴ In fact, volume-dependance is such a significant factor that the device manufacturer does not recommended use of this device when sample volume is <6 μL due to high false negatives. This makes this method of testing less useful in tear fluid analysis for dry eye patients who have less available tear fluid.²⁵

Use of electrochemical sensors as described herein provide a quantitative, accurate, numeric result that eliminates the variability as seen in lateral flow devices. Furthermore, the results of the impedance sensor show superior limit of detection, clinical correlation and accuracy. The results of this study show a sensitivity measurement of 70% with the impedance sensor as compared to lateral flow testing of 30%. This electrochemical sensor can provide results that are similar to results seen with in vitro enzyme-lined immunoassay (ELISA) for MMP-9.²⁷ A transition to this method of testing would decrease variability created by the limits in lateral flow testing and improve understanding of the clinical relevancy of this biomarker.

Elimination of the device variability and improvement on the sensitivity and limit of detection are critical factors to be able to follow disease improvement with MMP-9 testing. Changes in MMP-9 with treatment with ocular drops have been recorded at levels between 4.5 ng/mL and 48.5 ng/mL.¹⁶ The lateral flow device would not be able to reliably measure a change after treatment; however, the impedance-based sensor showed a limit of detection of 5 ng/mL and low standard error across this clinically relevant range that would make it useful for disease monitoring.²⁸ These novel biosensors are able to accurately measure contrived tear film samples from 5 ng/mL up to 75 ng/mL with low standard error. In addition, reports have shown that MMP-9 is highly concentration in the tear film upon awakening and then normalizes during mid-day.²⁹ Because of this diurnal curve, an objective device is needed to adequately classify patients and establish a baseline level for each individual. This level can then be compared across different time points for the same patient and compared to the contralateral eye to add validity to the measurements. We plotted the concentration of MMP-9 results in our patient cohort across different times throughout the day.

As for tear osmolarity, impedance sensors showed similar ability to separate dry eye from control subjects as published results for the tear osmolarity test. In this study the commercially available device for tear osmolarity did not detect high levels in any of the patients tested. The readings on the tear osmometer were all under 303 which is under the cutoff level for dry eye diagnosis. The test was performed by trained technicians following the manufacturer's recommendations following the standard clinic protocols for work ups for patients seen in the clinic. Due to masking of this study, the collection of these samples were not observed by the research team.

Limitations of this study include that 18% of the control subjects were there for post-operative evaluation. Their recent surgery and use of prescribed medications may have impacted their results. Also, there could be other subjects in the control group that had undiagnosed dry eye. This might contribute to the overlap seen between the dry eye and control subject. The tear film samples were frozen and stored prior to testing with the impedance sensor and studies have not been performed to assess the stability of the tear sample in these conditions. In addition, due to the small volume of tear collected using the microcapillary tubes, there was only enough fluid to run a single test on each biosensor and; therefore, duplicate or triplicate testing that is typically performed on new instrumentation to confirm results could not be done in this study.

In conclusion, tear MMP-9 and osmolarity levels are used in clinics to evaluate patients with dry eye disease. The use of these devices has changed the standard for evaluation, diagnosis and management of patients with dry eye as seen with the adoption of these tests in the work up for pre-operative patients.¹⁰ But limitations in the currently available devices has reduced the use of these tests in clinic. Having access to a quantitative measurement for MMP-9 is necessary for accurate clinical diagnosis as seen with the results in this study that show improved correlation to clinical exam findings and superior sensitivity of the test. In addition, having a device, such as the novel biosensor, that can provide measurements within the clinically relevant range of 5 ng/mL to 75 ng/mL, is essential to be able to study patients over time and in regard to their response to treatment. Overall, the most critical current need in tear film biomarker detection is for reduce variability. The best step forward is to introduce technology that can reduce this variability. The variance in results caused by the inherent limitations of lateral flow testing highlights the inferiority of this method of testing in tear film analysis and underscores the fact that that better technology approaches, such as seen with the use of electrochemical platforms should be the focus of future innovations.

### REFERENCES

1. Gayton J L. Etiology, prevalence, and treatment of dry eye disease. Clin Ophthalmol. 2009;3(1):405. doi:10.2147/OPTH.S5555
2. O'Neil E C, Henderson M, Massaro-Giordano M, Bunya V Y. Advances in Dry Eye Disease Treatment. Curr Opin Ophthalmol. 2019;30(3):166. doi:10.1097/ICU.0000000000000569
3. Farrand K F, Fridman M, Stillman I O″ Z, Schaumberg D A. Prevalence of Diagnosed Dry Eye Disease in the&nbsp;United States Among Adults Aged 18 Years and Older. Annu Meet Am Acad Ophthal-mology. 2017. doi:10.1016/j.ajo.2017.06.033
4. Pflugfelder S C, Solomon A, Stern M E. The Diagnosis and Management of Dry Eye A Twenty-five-Year Review. Cornea. 2000; 19(649):644-649.
5. Lemp M A, Baudouin C, Baum J, et al. The Definition and Classification of Dry Eye Disease: Report of the Definition and Classification Subcommittee of the International Dry Eye Workshop (2007). Ocul Surf. 2007; 5(2):75-92. doi:10.1016/S1542-0124(12)70081-2
6. Craig J P, Nichols K K, Akpek E K, et al. TFOS DEWS II Definition and Classification Report. Ocul Surf. 2017; 15(3):276-283. doi:10.101 6/J.JTOS.2017.05.008
7. Pflugfelder S C, Bian F, De Paiva C S. Matrix metalloproteinase-9 in the pathophysiology and diagnosis of dry eye syndrome. 2017:4-37. doi:10.2147/MNM.S107246
8. Epitropoulos A T, Matossian C, Berdy G J, Malhotra R P, Potvin R. Effect of tear osmolarity on repeatability of keratometry for cataract surgery planning. J Cataract Refract Surg. 2015; 41(8):1672-1677. doi:10.1016/J.JCRS.2015.01.016
9. Gupta P K, Drinkwater O J, VanDusen K W, Brissette A R, Starr C E. Prevalence of ocular surface dysfunction in patients presenting for cataract surgery evaluation. J Cataract Refract Surg. 2018; 44(9):1090-1096. doi:10.101 6/J.JCRS.2018.06.026
10. Starr C E, Gupta P K, Farid M, et al. An algorithm for the preoperative diagnosis and treatment of ocular surface disorders. J Cataract Refract Surg. 2019; 45(5):669-684. doi:10.1016/J.JCRS.2019.03.023
11. Tashbayev B, Utheim T P, Utheim Ø A, et al. Utility of Tear Osmolarity Measurement in Diagnosis of Dry Eye Disease. Sci Reports 2020 101. 2020; 10(1):1-7. doi:10.1038/s41598-020-62583-x
12. Chotikavanich, Suksri, et al. “Production and activity of matrix metalloproteinase-9 on the ocular surface increase in dysfunctional tear syndrome.” Investigative ophthalmology & visual science 50.7 (2009): 3203-3209.
13. Acera, Arantxa, Elena Vecino, and Juan A. Duran. “Tear MMP-9 levels as a marker of ocular surface inflammation in conjunctivochalasis.” Investigative ophthalmology & visual science 54.13 (2013): 8285-8291.
14. Leonardi, Andrea, et al. “Tear levels and activity of matrix metalloproteinase (MMP)-1 and MMP-9 in vernal keratoconjunctivitis.” Investigative ophthalmology & visual science 44.7 (2003): 3052-3058.
15. Lanza, Nicole L., et al. “The matrix metalloproteinase 9 point-of-care test in dry eye.” The ocular surface 14.2 (2016): 189-195.
16. Shetty, Rohit, et al. “Altered tear inflammatory profile in Indian keratoconus patients—The 2015 Col Rangachari Award paper.” Indian journal of ophthalmology 65.11 (2017): 1105.
17. Minařiková, Marcela, et al. “Tear matrix metalloproteinase-9 levels may help to follow a ocular surface injury in lagophthalmic eyes.” Plos one 17.9 (2022): e0274173.
18. Sakimoto, Tohru, and Mitsuru Sawa. “Metalloproteinases in corneal diseases: degradation and processing.” Cornea 31 (2012): S50-S56.
19. Garrana, R. M., et al. “Matrix metalloproteinases in epithelia from human recurrent corneal erosion.” Investigative Ophthalmology & Visual Science 40.6 (1999): 1266-1270.
20. Daniels, Julie T., et al. “Human corneal epithelial cells require MMP-1 for HGF-mediated migration on collagen I.” Investigative ophthalmology & visual science 44.3 (2003): 1048-1055.
21. Xue, Mei Lang, et al. “Regulation of MMPs and TIMPs by IL-1β during corneal ulceration and infection.” Investigative ophthalmology & visual science 44.5 (2003): 2020-2025.
22. Lam-Franco, Lorena, et al. “IL-1α and MMP-9 tear levels of patients with active ocular rosacea before and after treatment with systemic azithromycin or doxycycline.” Ophthalmic Research 60.2 (2018): 109-114.
23. Mathalone, Nurit, et al. “MMP expression in leaking filtering blebs and tears after glaucoma filtering surgery.” Graefe's Archive for Clinical and Experimental Ophthalmology 249 (2011): 1047-1055.
24. Hwa J, Id J, Lee H, Son J, Kim H. Importance of tear volume for positivity of tear matrix metalloproteinase-9 immunoassay. 2020. doi:10.1371/journal.pone.0235408
25. Koczula K M, Gallotta A. Lateral flow assays. Essays Biochem. 2016; 60:111-120. doi:10.1042/EBC20150012
26. Tomlinson A, Khanal S, Ramaesh K, Diaper C, McFadyen A. Tear Film Osmolarity: Determination of a Referent for Dry Eye Diagnosis. Invest Ophthalmol Vis Sci. 2006; 47(10):4309-4315. doi:10.1167/IOVS.05-1504
27. Benitez-del-Castillo J M, Soria J, Acera A, Munoz A M, Rodriguez S, Suarez T. Quantification of a panel for dry-eye protein biomarkes in tears: A comparative pilot study using standard ELISA and customized microarrays. Molecular Vision. 2021; 27:243-261.
28. Labriola L T, Campbell K G, Peacock M. Preclincial test results of matrix metalloproteinase in contrivted tear film using a novel impedance-based sensor. Unpublished. 2019
29. Markoulli M, Papas E, Cole N, Holden B A. The Diurnal Variation of Matrix Metalloproteinase-9 and Its Associated Factors in Human Tears. Invest Ophthalmol Vis Sci. 2012; 53(3):1479-1484. doi:10.1167/IOVS.11-8365

1. A biosensor for the detection of at least one analyte and osmolarity in a biological sample, the biosensor comprising: a carbon layer; at least one sensor electrode; ligands for the one or more analytes in contact with the carbon layer and/or the at least one sensor electrode, and a substrate.

2. The biosensor of claim 1, wherein the at least one sensor electrode is between the carbon layer and the ligands for the analyte.

3. The biosensor of claim 1, wherein the at least one analyte is MMP-9 and/or lacritin and the ligands for the one or more analytes are antibodies or aptamers that can specifically bind MMP-9 and/or lacritin.

4. The biosensor of claim 1, wherein osmolarity is detected in combination with any combination of analytes selected from MMP-9, lacritin, lacroferrin, lysozyme, calgranulin A/B, annexin A1, cystatin S, cathepsin S, PRP4 kinase, tear lipocalin, secretoglobin family 1D member 1 and 2, enolase 1alpha, mucin MUS5AC, nerve growth factor, human diamine oxidase, calcitonin gene-related peptides, neuropeptide Y, serotonin, epidermal growth factor, interleukins, chemokines, or albumin on the same biosensor or on a different biosensor.

5. The biosensor of claim 1, wherein the biosensor comprises a detection area for the at least one analyte and a detection area for osmolarity.

6. The biosensor of claim 5, wherein the detection area for the at least one analyte and the detection area for osmolarity are located on the same sensor electrode.

7. The biosensor of claim 5, wherein the detection area for the at least one analyte and the detection area for osmolarity are located on different sensor electrodes.

8. A method for detecting one or more analytes in a sample and osmolarity of the sample, comprising contacting the biosensor claim 1 with the sample and detecting the one or more analytes and the osmolarity with a detector.

9. The method of claim 8, wherein the detector detects binding or interaction of the one or more analytes and the ligands due to a change in electrical impedance caused by binding or interaction of the one or more analytes in the sample with the ligands.

10. The method of claim 8, wherein the sample is tears, tear film, aqueous layer of the tear film, aqueous humor, sweat, blood, serum, plasma, urine, saliva, or other bodily fluids.

11. A method for detecting one or more analytes in a sample and osmolarity of the sample, comprising contacting the sample with the biosensor of claim 1, and inserting the biosensor into or onto a device comprising (a) a detector to measure change in electrical impedance, and (b) a screen for visualizing the change in impedance, thereby detecting one or more analytes in the sample and the osmolarity of the sample.

12. The method of claim 11, wherein the amount of the one or more analytes in the sample is detected.

13. A method for detecting one or more analytes in a sample and osmolarity of the sample, comprising contacting the sample with the biosensor of claim 1, wherein the biosensor is present in a handheld device comprising (a) a detector to measure change in electrical impedance, and (b) a screen for visualizing the change in impedance, thereby detecting one or more analytes in the sample and the osmolarity of the sample.

14. The method of claim 13, wherein an amount of the one or more analytes in the sample is detected.

15. A method for diagnosing keratoconjunctivitis sicca (KCS) (dry eye), comprising contacting the biosensor of claim 1 with a tear, tear film, aqueous layer of the tear film, or aqueous humor sample of a subject, detecting an amount of one or more analytes in the sample and osmolarity of the sample, and diagnosing dry eye in the subject where the concentration of the one or more analytes in the sample is elevated or decreased as compared to a control sample or control standard and/or wherein the osmolarity of the sample is elevated as compared to a control sample or control element.

16. The method of claim 15, wherein the one or more analytes are MMP-9 and/or lacritin and wherein an increased amount of MMP-9 and/or decreased amount of lacritin as compared to a control indicates a diagnosis of dry eye.

17. The method of claim 15, wherein, an increased amount of osmolarity as compared to a control indicates a diagnosis of dry eye.

18. The method of claim 15, wherein, the one or more analytes are lacritin, lacroferrin, lysozyme, cystatin S, PRP4 kinase, mucin MUS5AC, calcitonin gene-related peptides, neuropeptide Y, MMP-9, calgranulin A/B, annexin A1, cathepsin S, lacritin, enolase 1 alpha, nerve growth factor, serotonin, interleukins, TNF alpha, albumin or change in epidermal growth factor, secretoglobin family 1D member 1 and 2, tear lipocalin or any combination thereof.

19. The method of claim 15, further comprising treating the subject with over-the-counter eye drops, over-the-counter moisturizing gels, over-the-counter ointments, prescription drops, cyclosporine (Restasis), lifitegrast (Xiidra), cyclosporine ophthalmic solution, avoidance of smoke, wind, and air conditioning, use of a humidifier, limiting screen time, wearing sunglasses, using tear duct (punctal) plugs, surgery to correct loose lower eyelids, or a combination thereof.

20. A handheld device comprising a biosensor for detection of one or more analytes and osmolarity comprising a carbon layer; at least one sensor electrode; ligands specific for the one or more analytes in contact with the carbon layer and/or the at least one sensor electrode, a substrate; and a detector connected to a data acquisition system.

21.-28. (canceled)
